(19) United States
(12) Patent Application Publication
(10) Pub. No.: US 2022/0016373 A1
(43) Pub. Date: Jan. 20, 2022

(54) AN IMPROVED SPEAKING VALVE

(71) Applicant: KAPITEX HEALTHCARE LIMITED, West Yorkshire (GB)

(72) Inventors: Ian David WORTHINGTON, West Yorkshire (GB); Alexander James BRIERLEY, West Yorkshire (GB)

(21) Appl. No.: 17/295,653

(22) PCT Filed: Nov. 22, 2019
(86) PCT No.: PCT/EP2019/082282

§ 371 Date: May 20, 2021

(30) Foreign Application Priority Data
Nov. 29, 2018 (GB) 1819427.4

(51) Int. Cl. A61M 16/04; A61M 16/20

(52) U.S. Cl. CPC A61M 16/0468; A61M 16/206

(57) ABSTRACT

A speaking valve for use with a tracheostomy and voice prosthesis, that, in a first configuration, allows inspiration and expiration through the valve; and in a second configuration resists expiration. Increasing the pressure difference across the valve by increasing the rate of expiration to a predetermined threshold causes the valve to transition into the second configuration; and the second configuration the valve continues to resist expiration until a predetermined negative pressure difference across the valve causes it to transition back into the first configuration.

### FIELD OF THE INVENTION

The invention relates to speaking valves, used to control the flow of air through a tracheostoma, and also to valve elements suitable for use in such speaking valves and elsewhere, especially in medical devices, and more particularly in medical airway management devices.

### BACKGROUND AND PRIOR ART

Speaking valves are known in themselves, and are used to control, or assist the control of the flow of air through a tracheostoma—a passage formed between the trachea of a human subject, and the outside air.

One particular, and typical use, will be described with reference to FIGS. 1 and 2. FIG. 1 shows a schematic cross-section through a human subject 1 who has undergone a laryngectomy. FIGS. 2A and 2B illustrate an enlargement of the region enclosed by the dashed-line circle. The removal of the larynx is often a consequence of throat cancer. Illustrated are the trachea 2, or windpipe, connecting the lungs to the tracheostoma 3, and also the oesophagus 4 connecting the stomach to the mouth 5. In order to allow a person to speak, in the absence of a larynx, a voice prosthesis 6 is fitted within a fistula made between the trachea 2 and the oesophagus 6. The voice prosthesis acts as a one-way valve, preventing food and drink within the oesophagus 6 reaching the lungs via the trachea 2. A speaking valve 7 is provided within the tracheostoma, between the outside air and the opening to the trachea 2. In its non-speaking configuration, the speaking valve 7 allows the flow of air into and out of the trachea as illustrated by the arrow in FIG. 2A, thereby allowing the individual to breathe.

When the individual wishes to speak, they take air into their lungs with the speaking valve 7 in the configuration shown in FIG. 2A, and then cause the valve 7 to close while exhaling the air. With the speaking valve 7 closed, air is forced through the voice prosthesis 6 and into the upper region of the oesophagus as indicated by the arrow in FIG. 2B. This flow of air vibrates soft tissue within the patient's oesophagus causing sound to be produced that the individual can control to produce speech.

In its simplest form, a speaking valve 7 could constitute an aperture through the tracheostoma that the individual can close by covering the aperture with a finger or thumb. In more advanced valves, a valve element occluding the aperture might be provided, biased into an open position. An individual can then push and hold the valve element into a closed position while speaking.

This interaction between a user's fingers and the speaking valve 7 (or indeed the tracheostomal opening itself) is unwanted because it draws attention to the use of the voice prosthesis, occupies the user's hands, and increases the risk of introducing micro-organisms into the trachea 2.

In order to address this problem, hands-free speaking valves have been developed, and one such variant is illustrated in FIGS. 3 and 4 in cross-section (A) and end-elevation (B) views. In this variant, the speaking valve comprises a tubular channel 9 that can be mounted in the tracheostoma. The interior of the channel 9 has a wall 10 extending across it, and the wall is provided with an aperture 11 through which air can pass, as illustrated by the arrow in FIG. 3A. A curved, flexible and resilient membrane 12 is affixed adjacent the aperture 11, so that, in the absence of force applied to the membrane 12, the aperture 11 remains unobscured, to allow a user of the valve to breathe.

When the user wishes to speak, they expel air with a much greater force than used for breathing, and the rush of air forces the membrane 12 to deform to the configuration shown in FIG. 4 such that it seals the aperture 11, thereby forcing air to pass through a voice prosthesis 6 as illustrated in FIGS. 1 and 2. As long as the air pressure within the user's trachea 2 remains relatively high, the membrane 12 remains in the closed position of FIG. 4. As soon as the air pressure within the trachea 2 drops below a threshold value, the resilience of the membrane 12 causes it to return to the open position of FIG. 3, stopping the flow of air through the voice prosthesis 6, and allowing breathing to continue via the aperture 11 in the speaking valve.

This configuration thereby allows hands-free operation of the speaking valve. However, in order for the valve to remain in the closed or “speaking” position shown in FIG. 4, the air pressure in the trachea needs to be maintained. This is quite tiring for a user, and does not allow them to pause while speaking, or leave gaps between words, because if they do so, the valve reverts to its open configuration. The user then needs to produce a further rush of air to close the valve again.

It is among the objects of the present invention to provide a solution to these and other problems.

### SUMMARY OF THE INVENTION

Accordingly, the invention provides a speaking valve to allow inspiration and controllable expiration through a tracheostoma, wherein, in a first configuration, said speaking valve allows inspiration and expiration through said valve; and in a second configuration said speaking valve resists expiration through said valve; and wherein in said first configuration, increasing the pressure difference across said valve by increasing the rate of expiration to a predetermined threshold expiration rate causes the valve to transition into a second configuration in which said valve resists expiration through said valve; and wherein in said second configuration said valve continues to resist expiration through said valve until a predetermined negative pressure difference across said valve causes the valve to transition back into said first configuration.

Preferably, in said second configuration, increasing the pressure difference across said valve above a predetermined threshold pressure causes said valve to transition into a third configuration in which said valve permits inspiration and expiration, and in which third configuration changes in expiration rate do not result in a change in valve configuration.

More preferably, said speaking valve is configured to allow it to be returned from said third configuration into either of said first or second configurations by manual intervention of a user.

In any aspect of the invention it is preferred that said predetermined threshold expiration rate is adjustable. Also in any aspect of the invention it is preferred that said predetermined negative pressure is adjustable.

In any aspect of the invention it is also preferred that the magnitude of the predetermined negative pressure is less than the magnitude of the pressure drop across the valve at the predetermined threshold expiration rate.

Where a third configuration of the valve is provided for, it is preferred that said predetermined threshold pressure is adjustable. It is also further preferred that a biasing mechanism is provided that, when the valve is in the said third configuration, maintains the valve in a state that permits inspiration and expiration.

The scope of the invention also includes a valve element suitable for use in a speaking valve disclosed herein, said valve element comprising a bistable diaphragm configured such that in one of its two stable configurations said diaphragm interacts with a valve seat to resist flow through said valve element.

Preferably, said valve element comprises a resiliently deformable diaphragm having a hole therethrough, mountable on a shaft having a diameter greater than the diameter of said hole. More preferably, said valve element further comprises an annular ridge located adjacent the perimeter of a face of said diaphragm, thereby improving interaction with said valve seat. More preferably still, said valve element comprises an annular ridge located adjacent the perimeter of each face of said diaphragm.

The invention also provides a valve element wherein said bistable diaphragm comprises a resiliently deformable diaphragm having a central hub and an annular rim wherein said hub and rim are joined by a web having a face that is not perpendicular to the axis of said annular rim.

Also included within the scope of the invention is an airway management device comprising a valve element disclosed herein.

### BRIEF DESCRIPTION OF THE FIGURES

The invention will be described with reference to the accompanying drawings, in which:

FIGS. 1 and 2 illustrate, in cross-section, the location of a speaking valve and voice prosthesis in a user;

FIGS. 3 and 4 illustrate a known speaking valve;

FIGS. 5-13 illustrate valve elements of the invention;

FIGS. 14-17 illustrate speaking valves of the invention;

FIGS. 18-20 illustrate, graphically, the pressure difference across a speaking valve in various configurations; and

FIGS. 21-22 illustrate, in exploded perspective view, a speaking valve of the invention.

### DESCRIPTION OF PREFERRED EMBODIMENTS

In this discussion of speaking valves, when we talk of a pressure difference across a valve, or across a diaphragm member of a valve, we shall define the pressure difference as being positive when the pressure on the tracheal side of the valve or diaphragm is greater than the pressure on the other side, i.e. typically atmospheric air pressure.

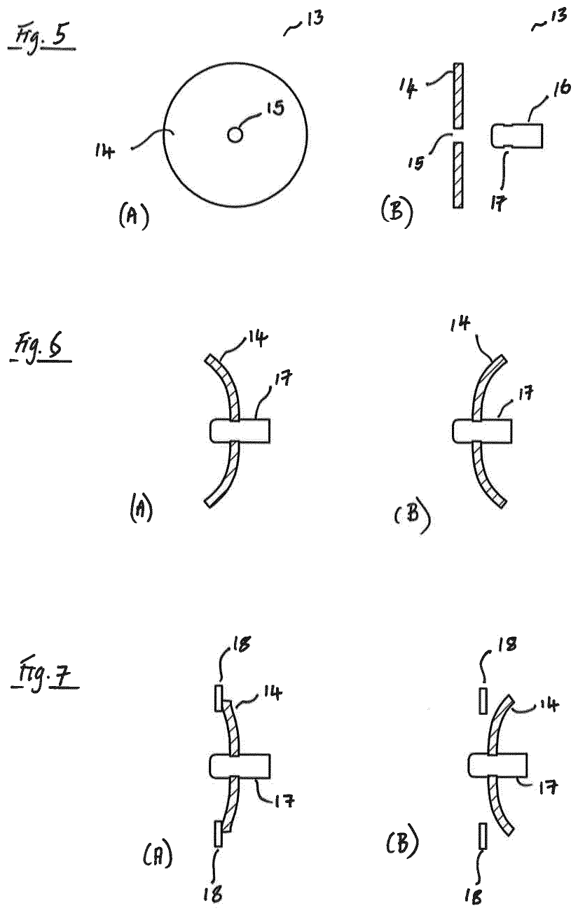

FIGS. 5-6 illustrate a valve element forming part of a speaking valve of the invention.

FIG. 5 illustrates in plan (A) and cross-sectional (B) views the operative part of a valve element, generally indicated by 13, forming part of a speaking valve of the invention. The valve element comprises a resiliently deformable diaphragm 14, having a through hole 15 in its centre. The diaphragm is mountable on a shaft 16 having a diaphragm-engaging region 17. The diameter of the diaphragm-engaging region of the shaft is made to be greater than the diameter of the hole 15 in the diaphragm 14. The diaphragm could be manufactured from a rubber-like material e.g. silicone rubber.

When the diaphragm 14 is mounted on the shaft 16, the difference in the two diameters causes the diaphragm 14 to deform, and to adopt one of the two stable positions illustrated in FIG. 6. In this configuration, the now bistable diaphragm 14 can be moved between each of the two stable positions by applying force to alternate faces of the diaphragm 14.

FIG. 7 illustrates how an annular sealing ring 18 may be positioned adjacent and parallel to one face of the diaphragm 14 such that, in a first position shown in FIG. 7A, an edge region of the diaphragm 14 abuts a face of the sealing ring 18. When mounted in a cylindrical passage forming part of a speaking valve, this configuration places the speaking valve into a closed, or “speaking” configuration. In the second configuration, illustrated in FIG. 7B, the edge region of the diaphragm 14, is spaced-apart from the sealing ring 18, thereby allowing air to pass between the diaphragm 14 and the sealing ring 18. When mounted in a cylindrical passage forming part of a speaking valve, this configuration places the speaking valve into an open, or “breathing” configuration.

By using the valve element construction illustrated in FIG. 7 within a speaking valve, a user can cause the valve to transition from the open, “breathing” configuration of FIG. 7B to the closed, “speaking” position of FIG. 7A by increasing their rate of expiration such that the pressure drop across the diaphragm 14 acting on the diaphragm's inner face causes the diaphragm to transition into the closed, or “speaking” position of FIG. 7A. Once this transition has occurred, the diaphragm will remain in the closed position even if the pressure difference is removed. As a result, the user does not have to maintain a positive pressure difference across the valve for it to remain in the speaking configuration. This therefore is less tiring, and allows a user to pause during speech without having the speaking valve return automatically to the breathing configuration.

When a user wishes to return the valve to the breathing configuration, they just need to create a negative pressure difference across the valve (by breathing in) sufficient to cause the diaphragm to transition back to the open configuration of FIG. 7B.

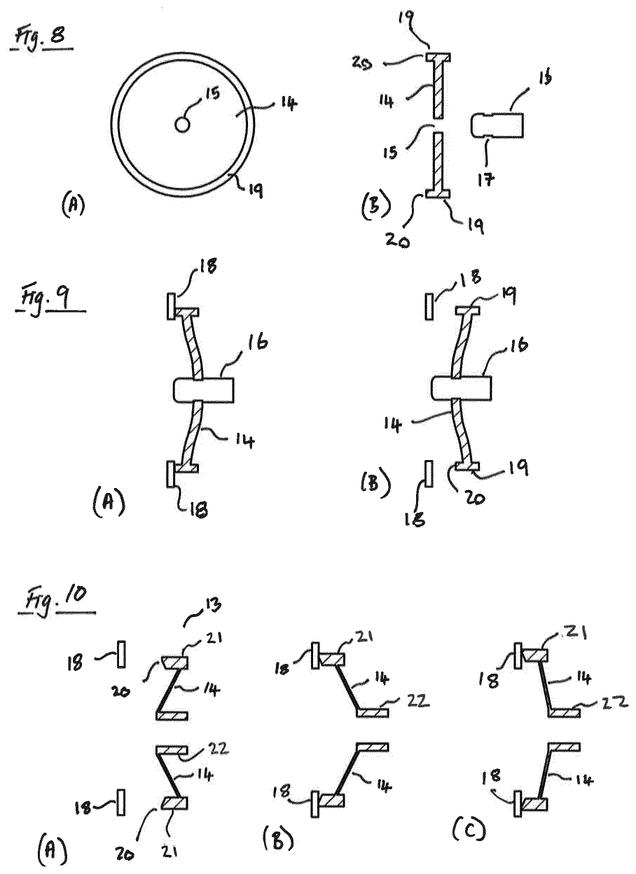

FIGS. 8 and 9 illustrate an alternative embodiment of a valve element 13 forming part of a speaking valve. FIGS. 8 and 9 correspond respectively to FIGS. 5 and 7 of the above embodiment, and like elements are numbered as above. As before, the hole 15 in the diaphragm 14 is of a smaller diameter than the diaphragm-engaging region 17 of the shaft 16. The diaphragm is again also made of a resilient material.

In this embodiment, the diaphragm 14 is provided with an annular ridge 19 adjacent the perimeter of the diaphragm, and extending away from the face of the diaphragm that is intended to face the sealing ring 18. The addition of this feature improves the seal between the diaphragm and the sealing ring, as the structure tends to keep the leading face 20 of the ridge at the same angle (e.g. parallel to face of the diaphragm at its mid-point).

In the embodiment illustrated, the annular ridge 19 is illustrated as being at the perimeter, but it could equally be located a little way in from the perimeter if desired. Also, the ridge 19 is illustrated as extending away from both faces of the diaphragm 14. Although this is not required for sealing, the symmetry ensures that the diaphragm 14 cannot be mounted the wrong way round on the shaft 16.

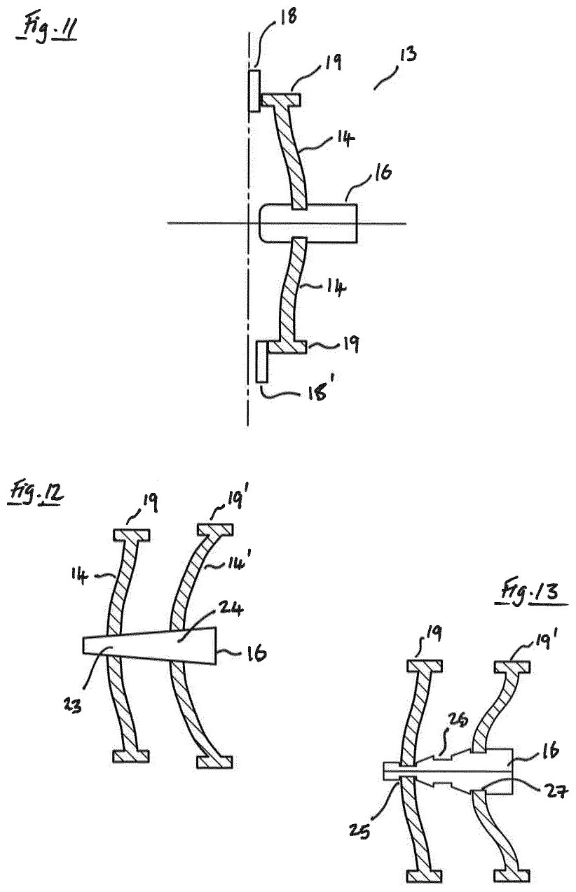

FIGS. 10A-10C illustrate, in cross-sectional view, an alternative embodiment of a valve element 13 forming part of a speaking valve of the invention. In this embodiment, the bistable diaphragm 14 is formed as a conical elastomeric diaphragm extending between an outer annular ring 21 and an inner support 22. In this embodiment, the inner support 22 is illustrated as also being annular, to enable it to be fitted over a shaft, but it could also be solid. FIG. 10A illustrates the valve element 13 in its unstressed configuration, being one of its two stable configurations. The outer annular ring 21 may be moved relative to the inner support 22 to place the valve element 13 in its second stable configuration, as illustrated in FIG. 10B. Also illustrated in FIGS. 10A-C is a sealing ring 18. In the configuration of FIG. 10A, the outer annular ring 21 is spaced-apart from the sealing ring 18, placing the valve element 13 into its “open configuration”. In FIG. 10B, a leading face 20 abuts the sealing ring 18, placing the valve element 13 into its “closed configuration”. In this embodiment, the leading face 20 of the outer annular ring 21 is angled, which provides a better seal with the sealing ring 18.

FIG. 10C illustrates a further configuration in which the sealing ring 18 is positioned closer to the valve element 13 than its “at rest” configuration of FIG. 10B. In this way, the force required to return the diaphragm 14 to its open configuration is reduced, and the seal between the outer annular ring 21 and the sealing ring 18 is improved. In a speaking valve, therefore, the position of the sealing ring 18 relative to the valve element 13 can be used to set the force required to open the valve to recommence normal breathing following speech production. Therefore, if the distance between the sealing ring 18 and the valve 13 was arranged to be adjustable, a user could select the force required (i.e. how hard they needed to breathe in) to open the speaking valve in this way.

FIG. 11 illustrates (in cross-section) this ability to change the force required to open the speaking valve after voice production. The top and bottom halves of FIG. 11 illustrate the sealing ring 18, 18′ being located in two different positions. In the top half, the sealing ring 18 is further away from the valve element 13 than the sealing ring 18′ illustrated in the bottom half. So, setting the sealing ring in the position illustrated in the bottom half of FIG. 11 would require a smaller force to open the valve than if it were positioned as shown in the top half of FIG. 11.

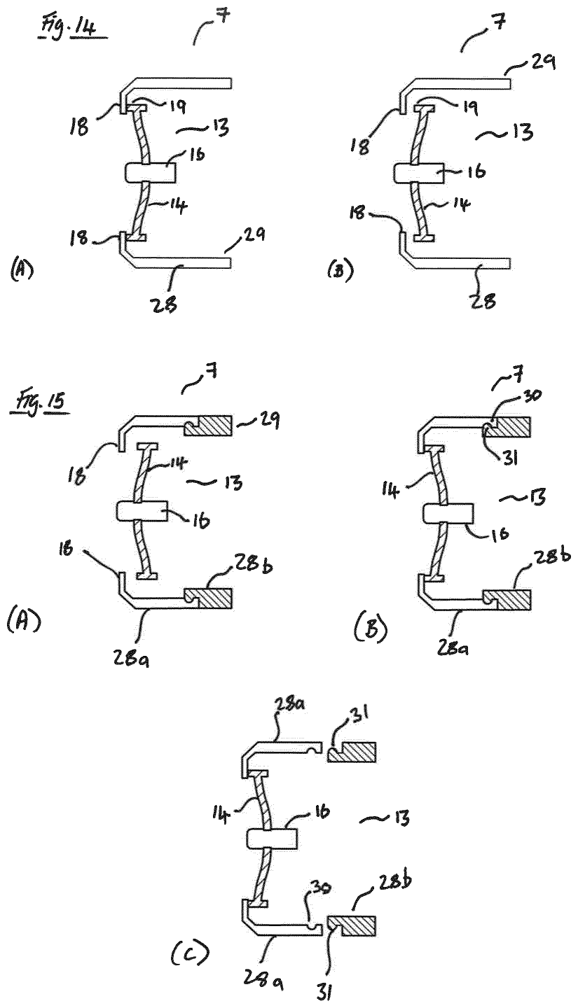

FIGS. 12 and 13 illustrate, in cross-sectional view, a means by which the force required to put the valve element 13 into its closed position may be adjusted. In this case, the valve element comprises a bistable membrane 14 as described above in which the bistability is created by mounting the membrane on a shaft 16 having a diameter greater than the diameter of the aperture 15 though the membrane 14. In FIG. 12, the shaft 16 is arranged to be tapered, so that if the membrane 14 is positioned in the shaft 16 at a region 23 having a smaller diameter by comparison to a region 24 having a larger diameter, the deformation of the membrane 14 increases, thereby increasing the force required to move it from its open position to its closed position. In this way, providing a mechanism whereby the relative position of the membrane 14 and the shaft can be adjusted, the required force to close the valve may be adjusted continuously.

In FIG. 13, the same principle is applied, but in this case the shaft 16 is provided with a number of discrete regions 25, 26, 27 comprising different diameter grooves in the shaft, so that a membrane 14 can be positioned in one of a number of grooves to set a required pressure required to close a valve.

FIG. 14 illustrates, in cross-sectional view an embodiment of a speaking valve 7 of the invention in closed (14A) and open (14B) configurations. This embodiment comprises a valve element 13 having a bistable membrane as described above. Like elements already described are numbered accordingly. The valve element 13 is located within an essentially cylindrical housing 28. The walls of the housing extend inwardly at the end of the valve furthest from the end 29 of the housing 28 intended to be connected to a tracheostoma forming a sealing ring 18 against which the annular ridge 19 of the valve element 13 can abut to form a seal. It will be evident, that alternative valve elements described herein can be mounted in a similar fashion to create a speaking valve.

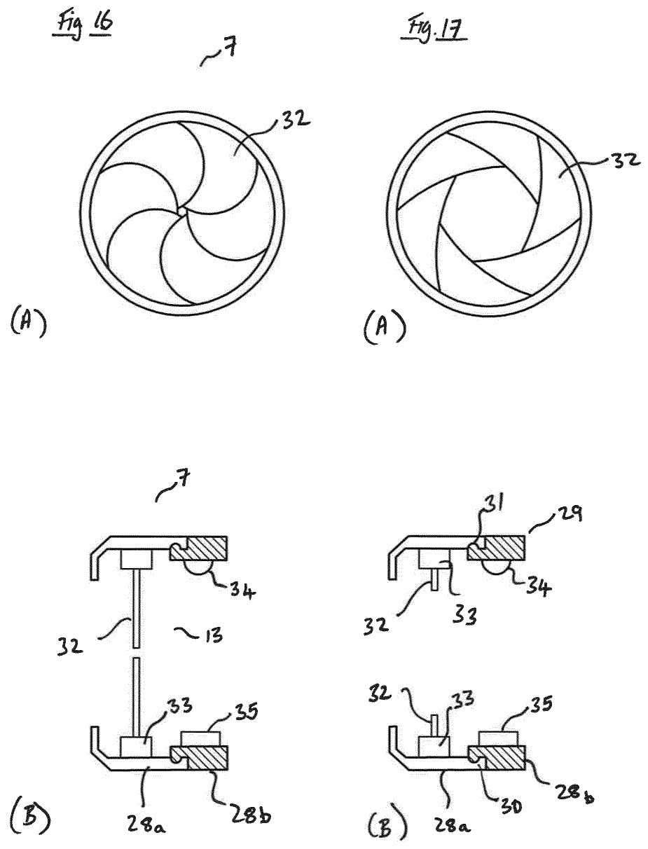

FIG. 15 illustrates, in cross-sectional view, a further embodiment of a speaking valve 7 of the invention having an additional feature. The valve is similar to that described in FIG. 14, and corresponding elements are again numbered accordingly. In this embodiment, the cylindrical housing 28 is formed of two parts, 28a and 28b that are releasably joined together. In the embodiment illustrated, the joining feature comprises a corresponding annular indent 30 and detent 31, formed of resilient material such that the two parts 28a, 28b can be releasably snapped together.

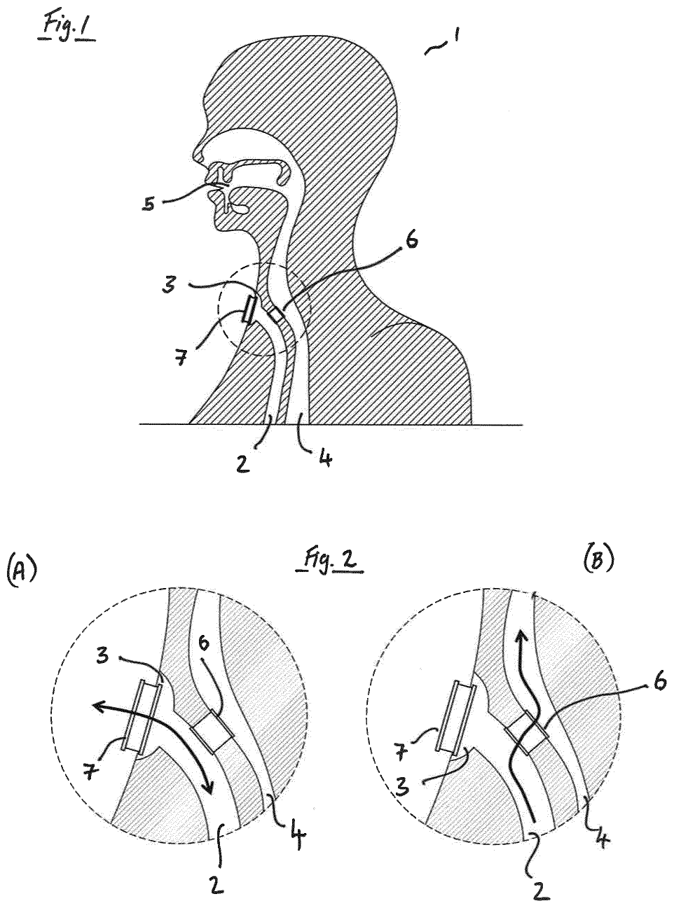

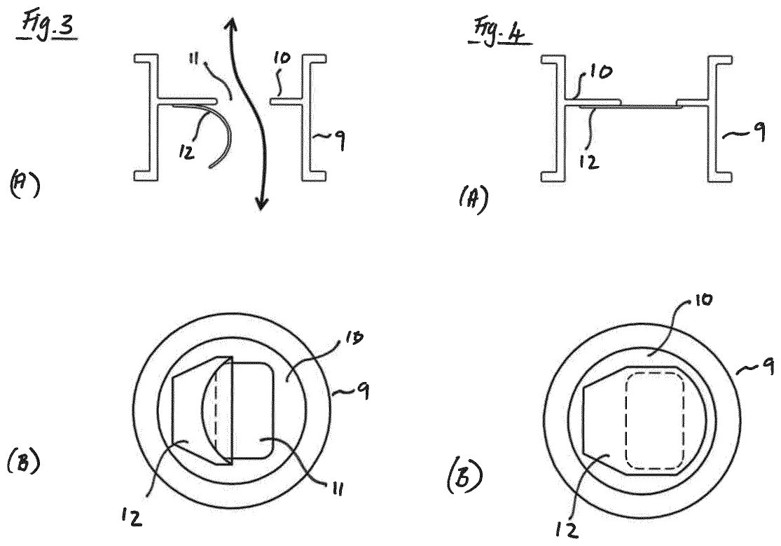

FIGS. 15A-15C illustrate three configurations of this speaking valve. In FIG. 15A, the valve element 13 is in its open position, allowing a user to breathe through their tracheostoma. If the user exhales more quickly than the normal breathing rate, thereby increasing the pressure drop across the membrane 14 above a threshold value, the membrane will be moved into its closed position, as illustrated in FIG. 15B. With the speaking valve now closed, the user can now speak, by forcing air through a voice prosthesis (as illustrated in FIGS. 1 and 2). Because the membrane 14 is in a stable configuration, the user does not have to maintain a positive pressure to keep the valve closed and can, e.g. pause during speech. When the user wishes to stop speaking, and resume normal breathing, they create a negative pressure across the membrane 14 by breathing in, returning the membrane to the open position illustrated in FIG. 15A.

While the valve is in the speaking configuration of FIG. 15B, if a user increases the pressure difference across the membrane 14 above a second threshold, for example if they cough, the two parts 28a, 28b of the valve will separate, into the configuration shown in FIG. 15C. The pressure difference required to transition into this configuration may be set by design of the releasable connection mechanism joining the two parts 28a and 28b.

In particularly preferred embodiments, the two parts 28a and 28b may be loosely tethered together so that should a user cough, and detach part 28a from part 28b, the outer part 28a is not lost. In even more preferred embodiments the two parts 28a and 28b may be biased apart from each other with a force less than the force required to separate the parts. In this way, should a user's breathing causes the two parts to separate, they will be held in a spaced-apart relationship, to ensure that subsequent breathing is uninterrupted. The user can then reconnect the two parts, as required.

FIGS. 16A-16B and FIGS. 17A-17B illustrate in elevation (A) and cross-sectional (B) view respectively, another embodiment of a speaking valve 7 of the invention. Again, like elements already described are similarly numbered. In this embodiment, the valve element 13 comprises an aperture closed by an iris arrangement 32 such as those found in camera mechanisms or iris valves. The iris 32 may be moved from a closed position as illustrated in FIG. 16 into an open position as illustrated in FIG. 17 by the action of an iris actuator 33. A pressure sensor 34 is also provided, to measure the pressure drop across the iris 32. A controller 35 is configured to receive a measurement of the pressure drop from the sensor 34 and actuate the iris to close when a predetermined pressure drop is achieved by the user, thereby allowing speech. A further predetermined pressure drop, or pattern of pressure can then be sensed by the sensor, and the controller configured to return the iris to its open position.

It will be appreciated that the use of a controller (e.g. an electronic controller) allows any pattern of pressure drop (i.e. breath) to be used to transition the iris between the open and closed positions. This could include opening the valve from the speaking position in response to an overpressure event such as might be caused by a cough. Optionally, the release mechanism comprising the two parts 28a and 28b as described above could also be used.

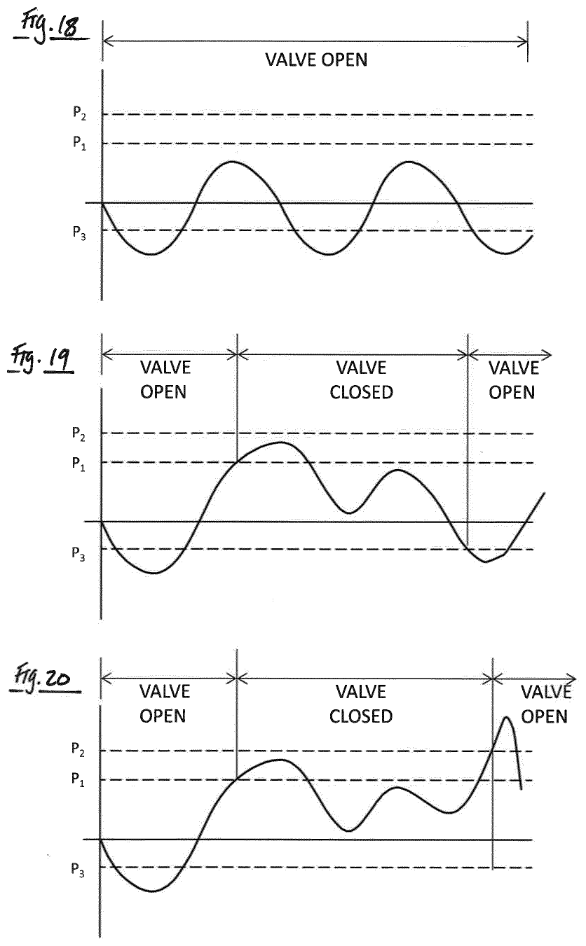

FIGS. 18-20 illustrate, in graphical form, the operation of a speaking valve of the invention. In each graph, the vertical axis represents the pressure drop across the membrane (or iris) in a speaking valve of the invention, and the horizontal axis represents time.

FIG. 18 represents normal breathing with the valve in the open position. The pressure difference never reaches the first predefined threshold P₁, and so the valve remains in the open position.

FIG. 19 represents the situation where the user increases the pressure drop above the first predefined threshold, P₁, which causes the valve to move into its closed configuration, and remain there, until the pressure drop reaches a predetermined negative pressure, P₃, when the valve moves back to its open configuration

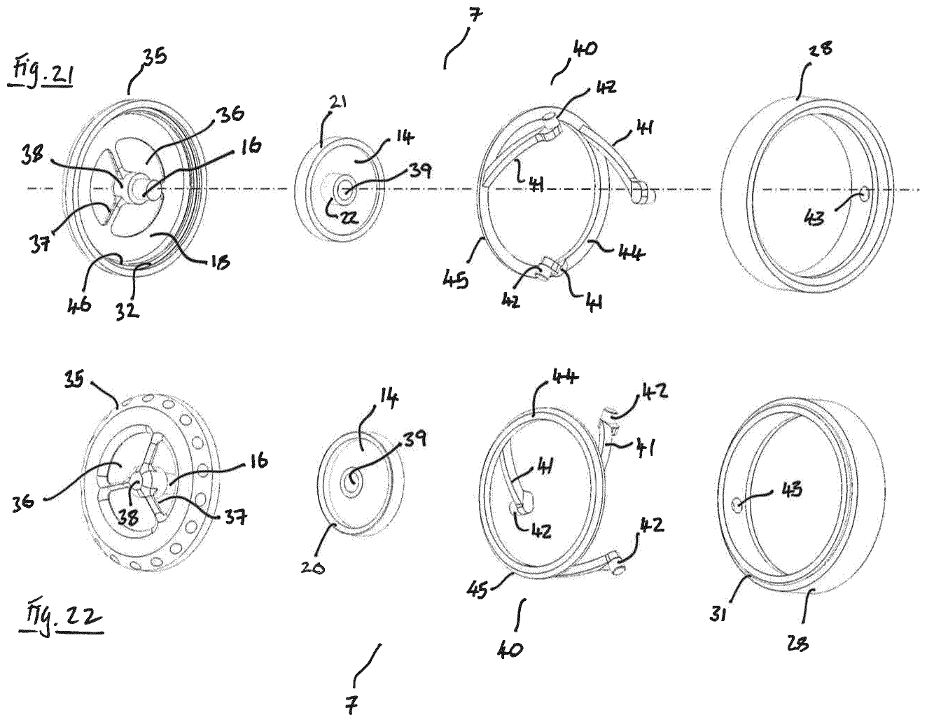

FIG. 20 represents the situation where the user increases the pressure drop above the first predefined threshold, P₁, which causes the valve to move into its closed configuration, and remain there, until the pressure drop reaches a predetermined a higher predetermined pressure difference P₂, which causes the valve to open, e.g. in the configuration shown in FIG. 15C, and remain in that configuration despite subsequent changes in breathing.

FIGS. 21 and 22 show exploded perspective views of a preferred embodiment of a speaking valve of the invention, general indicated by 7. The dashed line in FIG. 21 indicates the axis along which the parts have been exploded. FIG. 21 is illustrated looking obliquely towards the front end of the valve, and FIG. 22 is illustrated looking obliquely towards the rear, or tracheostomal, end 29 of the valve 7.

The valve comprises a generally circular front portion 35 having a hole 36 passing through it. The hole 36 is trisected by three arms 37 that support a central boss 38. Attached to the boss 38 is a reawardly-extending shaft 16. A portion of the interior surface of the front portion 35 comprises the sealing ring 18 against which the leading face 20 of the outer annular ring 21 of the diaphragm 14 can abut and seal. This bistable diaphragm is as illustrated in FIG. 10, but other valve elements described herein could also be used. An inner support 22 is provided at the centre of the diaphragm 14, that is furnished with a hole 39 allowing it to be mounted on the shaft 16.

A housing 28 is also provided, having a circumferential detent 31 adapted to reversibly mate with a circumferential indent 30 on the front portion 35. This arrangement is as described in the embodiments of FIGS. 15 and 16.

Also provided is a spring element 40 having three resiliently deformable legs 41 extending from a ring 44. At the end of each leg 41 is an outwardly-facing lug 42 so sizes and positioned as to rotatably fit within corresponding lug holes 43 provided on the inside face of the housing 28. Once assembled, the outer edge 45 of the ring 44 clips within a circumferential groove 46 in the front portion 35 to hold it in place. This spring element 40 constitutes the biasing arrangement discussed in the description of the embodiment of FIG. 15.

1. A speaking valve to allow inspiration and controllable expiration through a tracheostoma, wherein, in a first configuration, said speaking valve allows inspiration and expiration through said valve; and in a second configuration said speaking valve resists expiration through said valve; and
wherein in said first configuration, increasing the pressure difference across said valve by increasing the rate of expiration to a predetermined threshold expiration rate causes the valve to transition into a second configuration in which said valve resists expiration through said valve; and
wherein in said second configuration said valve continues to resist expiration through said valve until a predetermined negative pressure difference across said valve causes the valve to transition back into said first configuration.

2. A speaking valve according to claim 1 wherein in said second configuration, increasing the pressure difference across said valve above a predetermined threshold pressure causes said valve to transition into a third configuration in which said valve permits inspiration and expiration, and in which third configuration changes in expiration rate do not result in a change in valve configuration.

3. A speaking valve according to claim 2 wherein said speaking valve is configured to allow it to be returned from said third configuration into either of said first or second configurations by manual intervention of a user.

4. A speaking valve according to claim 1 wherein said predetermined threshold expiration rate is adjustable.

5. A speaking valve according to claim 1 wherein said predetermined negative pressure is adjustable.

6. A speaking valve according to claim 1 in which the magnitude of the predetermined negative pressure is less than the magnitude of the pressure drop across the valve at the predetermined threshold expiration rate.

7. A speaking valve according to claim 2 wherein said predetermined threshold pressure is adjustable.

8. A speaking valve according to claim 2 wherein a biasing mechanism is provided that, when the valve is in the said third configuration, maintains the valve in a state that permits inspiration and expiration.

9. A valve element suitable for use in a speaking valve according to claim 1, said valve element comprising a bistable diaphragm configured such that in one of its two stable configurations said diaphragm interacts with a valve seat to resist flow through said valve element.

10. A valve element according to claim 11 comprising a resiliently deformable diaphragm having a hole therethrough, mountable on a shaft having a diameter greater than the diameter of said hole.

11. A valve element according to claim 10 further comprising an annular ridge located adjacent the perimeter of a face of said diaphragm, thereby improving interaction with said valve seat.

12. A valve element according to claim 11 comprising an annular ridge located adjacent the perimeter of each face of said diaphragm.

13. A valve element according to claim 9 wherein said bistable diaphragm comprises a resiliently deformable diaphragm having a central hub and an annular rim wherein said hub and rim are joined by a web having a face that is not perpendicular to the axis of said annular rim.

14. An airway management device comprising a valve element according to claim 9.
